(19) United States
(12) Patent Application Publication
(10) Pub. No.: US 2011/0257108 A1
(43) Pub. Date: Oct. 20, 2011

(54) SYNTHESIS AND ACTIVITY OF LACTOSE ESTERS

(75) Inventor: Marie K. Walsh, North Logan, UT (US)

(73) Assignee: Utah State University, North Logan, UT (US)

(21) Appl. No.: 12/760,280

(22) Filed: Apr. 14, 2010

(51) Int. Cl. A01N 43/16; A01P 1/00; C12P 19/44; C13K 5/00

(52) U.S. Cl. 514/25; 536/18.2; 435/74

(57) ABSTRACT

This disclosure provides for a novel lactose monolaurate (LML) with the structure

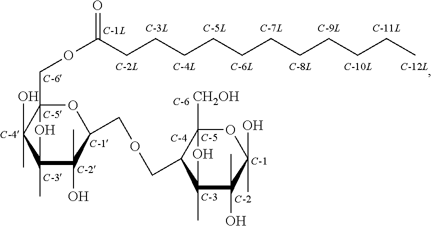

useful as an antimicrobial agent and as a potential substitute for other sugar esters. Methods of synthesizing LML using immobilize lipases and various solvents are also provided.

This application claims the priority of U.S. Provisional Application Ser. No. 61/168,995 entitled “SYNTHESIS AND ACTIVITY OF LACTOSE ESTERS” filed on Apr. 14, 2009, the entire contents and substance of which are hereby incorporated by reference herein.

### STATEMENT REGARDING FEDERALLY SPONSORED RESEARCH OR DEVELOPMENT

Not applicable

### NAMES OF THE PARTIES TO A JOINT RESEARCH AGREEMENT

Not applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

1. Field of Invention

The present invention is in the technical fields of Esters and Antimicrobials.

2. Description of the Related Art

The enzymatic synthesis of sugar esters has been investigated for over 20 years and is typically preferred to the chemical synthesis since it is more specific and conducted under milder conditions. Uses of sugar esters, which are characterized as non-ionic biodegradable surfactants, vary and depend on the characteristics of the substrates (sugar and lipid). Typical applications are as emulsifiers for personal care products, medical supplies, and in foods and as antimicrobial agents.

The different conditions used for the synthesis of sugar esters are multitude and include the type of solvent, ratio of sugar to lipid, the specific sugar and lipid, temperature, and type of immobilized lipase. To optimize yield during synthesis, various solvents (2-methyl-2-butanol (2M2B), acetone, hexane, and methyl ethyl ketone (MEK)) have been investigated, typically with the addition of molecular sieves for water removal, which is generated during the esterification reaction of a sugar and fatty acid. Water plays an important role in the equilibrium of the reaction, with limited water favoring the esterification reaction, while resulting in limited solubility of the sugar and eventual inactivation of the lipase. Solvents that can dissolve both sugars and lipids include dimethyl sulfoxide (DMSO), pyridine, and dimethylformamide, but these solvents often inactivate the lipase and are incompatible with food applications. To overcome this solubility issue, reaction conditions in supercritical acetone, supercritical carbon dioxide, DMSO in 2M2B, and ionic liquid have been investigated.

The ratio of sugar to lipid used typically varies from equal to ratios where the sugar is in excess or the lipid is in excess. The typical range of sugar to lipid ratio in the literature is from 3:1 to 1:3. The types of lipids that have been used include the fatty acids from four to sixteen carbons and virtually most known mono- and di-saccharides. The use of vinyl or methyl lipids as the substrate is also common with the use of vinyl lipids resulting in greater yields. The esterification of fatty acids to sugars results in the production of water while the transesterification with vinyl lipids results in acetaldehyde. Since water is non-toxic, the use of the fatty acids may be preferred depending on the application.

Temperatures used for esterification reactions ranges from 50 to 80° C. with the immobilized form of the lipase generally being more temperature stable than the free form. Immobilized lipases are generally more active at temperatures of 50-70° C. The types of immobilized lipases used include the lipase from Thermomyces lanuginosus (TL), Pseudomonas cepacia (PC), Mucor miehei (MM), and Candida antarctica (CA). CA and PC lipases are non-specific and TL and MM lipases are sn-1,3 specific with respect to triacylglycerol hydrolysis. The concentrations of immobilized lipases for esterification in batch reactions generally range from 0.1% to 10% with some researchers using immobilized lipase reactors for continuous ester production. Lipase from Candida antarctica is commonly used for the synthesis of sugar esters. The concentration of lipase influences the initial rate, but may not affect the equilibrium state of the reaction, which is generally measured in days.

In regards to solvents, synthesis of glucosylmyristate with CA has been shown to be dependent on the solvent with the highest yields in 2M2B, followed by acetone, hexane and finally diethylether. Other studies have also shown that high ester yields are obtained in 2M2B.

RSM is a very useful statistical technique for complex processes and has been applied previously to optimize the synthesis of lipase-catalyzed reactions.

### BRIEF SUMMARY OF THE INVENTION

This invention provides for the novel lactose monolaurate (LML) compound of FIG. 3, which is

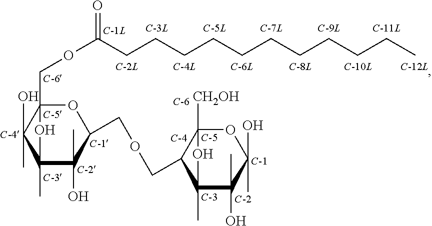

and methods of synthesizing the lactose monolaurate compound of FIG. 3, and various methods of using the lactose monolaurate compound of FIG. 3. Without limiting the invention in anyway, the novel lactose monolaurate has utility as an antimicrobial agent, and may find additional utility in uses common to sugar esters, including, but not limited to, utility as an emulsification agent.

### DEFINITIONS

“HPLC” means high performance liquid chromatography (synonymous with high pressure liquid chromatography).
“LML” means the novel lactose monolaurate disclosed in this application and shown in FIG. 3. The terms “LML,” “lactose monolaurate,” and “lactose lauryl esters” are used interchangeably in this application.
“SML” means sucrose monolaurate
“CA” means lipase from Candida antarctica
“PC” means lipase from Pseudomonas cepacia
“MM” means lipase from Mucor miehei
“TL” means lipase from Thermomyces lanuginosus
“2M2B” means 2 methyl-2-butanol
“BHI” means brain heart infusion
“LB” means lauria-bertani
“MEK” means methyl ethyl ketone
“RSM” means response surface methodology
“mmol/hr/g enz” means milimole per hour per gram lipase
“DMSO” means dimethyl silfioxide
“μ” means micro as in microliter (μL)

### BRIEF DESCRIPTION OF THE DRAWINGS

FIG. 1. Synthesis rates of lactose monolaurate (LML) (FIG. 1A) and sucrose monolaurate (FIG. 1B) with the immobilized lipase from Thermomyces lanuginosus (TL) in 2M2B (▪); Candida antarctica (CA) in 2M2B (◯); Mucor miehei (MM) in 2M2B (); Pseudomonas cepacia (PC) in 2M2B (▴). The y-axis shows the amount of LML or SML (monoester) synthesized in milligrams per milliliter (mg/ml). The x-axis shows time of synthesis in days.

FIG. 2. Lactose and sucrose solubilities in various solvents. The y-axis shows the solubility of lactose and sucrose in micromols per liter of solution. The x-axis shows the particular solvents used, including MEK, acetone, acetonitrile, and 2M2B. Dark bars represent lactose solubility and light bars represent sucrose solubility.

FIG. 3. The atom numbering scheme for the structure of the novel lactose monolaurate (LML). Analysis of the 13C NMR features of the purified LML esters synthesized by TL, MM and PC revealed that the LML products were all esterified at the C6 prime carbon (C6′) with lactose primarily in the alpha configuration. “L” refers to a carbon derived from a lipid substrate.

FIG. 4. HPLC chromatograms of lactose ester reactions with various lipases and solvents. (FIG. 4A) Reaction in acetone with lipase from Thermomyces lanuginosus (TL) after 7 days. (FIG. 4B) Reaction in 2M2B with lipase from Mucor miehei (MM) after 3 days. (FIG. 4C) Reaction in 2M2B with lipase from Pseudomonas cepacia (PC) after 14 days. (FIG. 4D) Reaction in 2M2B with lipase from Candida antarctica (CA) after 9 days. Identified peaks; 1, lactose (2.2 min), 2, lactose monoester (LML) (6.8-7.9 min), 3, lauric acid (11.4 min). Peaks sharing the same letter have the same retention times.

FIG. 5. HPLC chromatograms of sucrose ester synthesis with various lipases in 2M2B. (FIG. 5A) Reaction with lipase from Thermomyces lanuginosus (TL) after 10 days. (FIG. 5B) Reaction with lipase from Mucor miehei (MM) after 14 days. (FIG. 5C) Reaction with lipase from Pseudomonas cepacia (PC) after 14 days. (FIG. 5D) Reaction with lipase from Candida antarctica (CA) after 8 days. Identified peaks; 1, sucrose (2.2 min), sucrose monoester (6.8-7.9 min), 3 lauric acid (11.4 min). Peaks sharing the same letter have the same retention times.

FIG. 6. Response surface plots showing the mutual effects of substrate ratios with temperature ((FIG. 6A) at a constant lipase concentration of 32 mg/mL) and with lipase concentration ((FIG. 6B) at a constant temperature of 61° C.) on the synthesis of lactose monolaurate in 2M2 M with Mucor miehei (MM) lipase.

FIG. 7. Microbial growth inhibition of selected bacteria at concentrations of LML ranging from 0.001 to 0.10%. Leftmost bar indicates inhibition with 0.001% LML. Second leftmost bar indicates inhibition with 0.005% LML. Third leftmost bar indicates inhibition with 0.01% LML, and the rightmost bar indicates inhibition with 0.1% LML. The y-axis shows the percent growth inhibition. The x-axis shows the particular bacteria tested for susceptibility to LML.

FIG. 8. Inhibition of Listeria monocytogenes strain J177 by LML. The y-axis shows percent growth inhibition and the x-axis shows concentration of LML used.

FIG. 9. Inhibition of Listeria monocytogenes strain N1 227 by LML. The y-axis shows percent growth inhibition and the x-axis shows concentration of LML used.

FIG. 10. Inhibition of Listeria monocytogenes strain N3013 by LML. The y-axis shows percent growth inhibition and the x-axis shows concentration of LML used.

FIG. 11. Inhibition of Listeria monocytogenes strain R2499 by LML. The y-axis shows percent growth inhibition and the x-axis shows concentration of LML used.

FIG. 12. Inhibition of Listeria monocytogenes strain C056 by LML. The y-axis shows percent growth inhibition and the x-axis shows concentration of LML used.

### DETAILED DESCRIPTION OF THE INVENTION

The following materials, methods, embodiments, examples and disclosures may be useful in the practice of the present invention. The various heading are provided for ease of reading and in no way limit the invention.

### Materials

Vinyl laurate (226.4 g/mol), sucrose (324.3 g/mol), molecular sieves (3 Å), lipase acrylic resin from C. antarctica (CA) (Lot#047K1672), Amano Lipase PS-C I (from P. cepacia) (PC) (Lot#07703EE)), Lipozyme, immobilized from M. miehei (MM) (Lot#1285317), deuterated DMSO, and lauric acid were from Sigma-Aldrich (St. Louis, Mo., USA). Novozyme lipase from T. lanuginosus (TL) (Lot#35001701) was from Codexis (Redwood City, Calif., USA), and lactose (324.3 g/mol) was from Proliant (Ames, Iowa, USA). Nylon syringe filters (0.2μ) and solvents (acetonitrile, acetone, MEK, and 2M2B) were from Fisher Scientific (Pittsburgh, Pa., USA).

### Synthesis of LML, Lactose Lauryl Esters and Sucrose Lauryl Esters Using Immobilized Lipases.

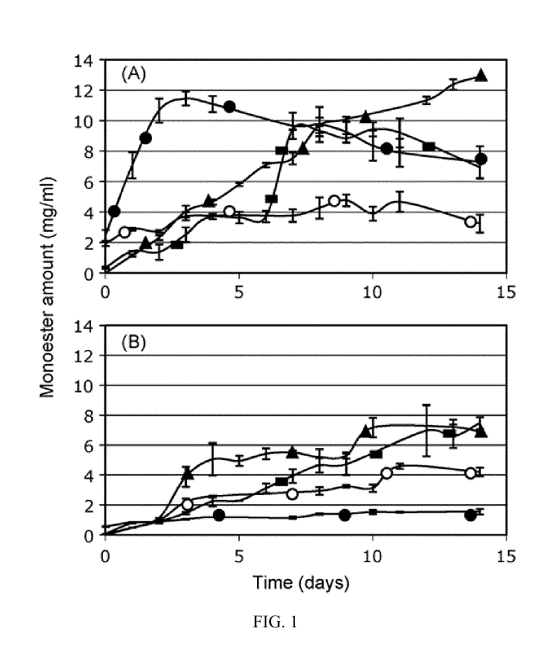

Referring now to FIG. 1, four different immobilized lipases (CA, PC, MM, and TL) were used to synthesize LML and SML using the same temperature, lipase (CA, PC, MM, or TL), vinyl laurate, and sugar concentrations with three different solvents (2M2B, acetone, and MEK). The amount of the monoester (LML or SML) synthesized over time (days) was determined via high-performance liquid chromatography (HPLC) with a standard curve. The rate and yield of the monoester produced was determined for each lipase/solvent/sugar combination. One specific combination of lipase/solvent (MM in 2M2B) which had a high yield was optimized for LML synthesis, using RSM. The solubilities of lactose and sucrose in each solvent were also determined. All reactions were conducted in triplicate and data expressed as means with standard error values unless noted.

Referring to FIG. 1 in more detail, there is shown the amount of LML (FIG. 1A) and SML (FIG. 1B) synthesized overtime (14 days) for synthesis of lactose monolaurate (LML) (FIG. 1A) and sucrose monolaurate (SML) (FIG. 1B) with the immobilized lipase from Thermomyces lanuginosus (TL) in 2M2B (▪); Candida antarctica (CA) in 2M2B (◯); Mucor miehei (MM) in 2M2B (); Pseudomonas cepacia (PC) in 2M2B (▴). The synthesis of LML and SML was conducted at constant lipase, temperature (55° C.), and substrate concentrations. It is desirable to stay below the evaporation temperatures of the solvents, the lowest of which was acetone, which has a boiling point of 56.5° C. at ambient pressure. Several points can be made from the graphs in FIG. 1. The highest monoester yields were obtained with lactose and it was possible to determine the synthesis rate based on the time of maximum ester synthesis. An example is the synthesis of LML in FIG. 1A with MM in 2M2B, which shows a maximum at day 3, compared to the continued production of LML and SML by PC in 2M2B over the 14 day time period. In contrast to monoester yield with PC in 2M2B, most lipase/solvent combinations reached a maximum amount of monoester in about 10-14 days with some showing a decrease in monoester content (e.g. FIG. 1A, MM and TL in 2M2B) over the time course that will be discussed below.

Table 1 shows the percent monoester yields (LML and SML) and rates for each of the lipase/solvent combinations used. The maximum theoretical yield was 22 mg/mL, based on the amount of the limiting reactant (sugar). Overall, the solvent 2M2B showed the highest yields and reaction rates for both LML and SML synthesis except for TL in acetone in which LML synthesis was slightly higher. Based on the data in Table 1, the solvent MEK was the least effective for each of the lipase/solvent combinations. With respect to the lipases, PC and TL showed the highest yields with sucrose and PC followed by MM and TL showed the highest yields with lactose. CA showed similar yields and rates with sucrose and lactose. PC was also similar with both sugars in MEK and acetone, as was TL in 2M2B. The lowest yields were obtained using MM with sucrose, and using CA with lactose, depending on the solvent used. Lipase from CA was found to be the least effective for LML synthesis depending on the solvent. SML yields for this lipase were not as low as from MM. Specifically for LML synthesis, MM in 2M2B had the highest reaction rate, as indicated by the shortest reaction time of 3 days.

TABLE 1
Reaction rates and yields of SML and LML.

| | | | | LML rate | SML rate |
|---|---|---|---|---|---|
| Enzyme (Lipase) | Solvent | % LML | % SML | (mmol/h/g enz) | (mmol/h/g enz) |
| TL from | 2M2B | 35.5 ± 3.10 | 32.9 ± 2.71 | 4.4 | 4.9 |
| Thermomyces | Acetone | 43.1 ± 0.08 | 13.2 ± 0.66 | 5.9 | 2.4 |
| lanuginosus | MEK | 12.0 ± 0.01 | 1.6 ± 0.17 | 1.8 | 0.29 |
| MM from | 2M2B | 52.4 ± 1.88 | 7.2 ± 0.78 | 26.7 | 0.81 |
| Mucor | Acetone | 32.5 ± 2.06 | 0.8 ± 0.35 | 13.8 | 0.31 |
| miehei | MEK | 12.2 ± 1.72 | 0 | 1.3 | 0 |
| PC from | 2M2B | 56.6 ± 1.45 | 34.2 ± 1.66 | 10.4 | 4.8 |
| Pseudomonas | Acetone | 16.8 ± 0.21 | 10.0 ± 3.45 | 3.6 | 1.9 |
| cepacia | MEK | 1.3 ± 0.31 | 1.3 ± 0.31 | 0.89 | 0.13 |
| CA from | 2M2B | 21.8 ± 1.57 | 20.9 ± 0.72 | 3.1 | 2.9 |
| Candida | Acetone | 1.3 ± 0.14 | 3.6 ± 0.51 | 0.17 | 1.3 |
| antarctica | MEK | 0.6 ± 0.15 | 0.5 ± 0.12 | 0.10 | 0.10 |

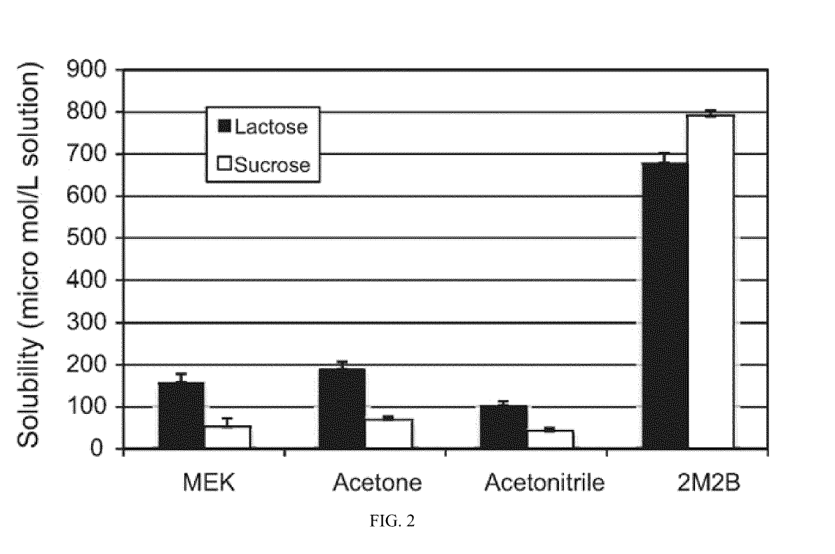

PC in 2M2B actually showed a slightly higher yield (56.6%) than MM in 2M2B (52.4%), but the rate is much slower due to the length of time (14 days) to reach maximum yield. Specifically for SML synthesis, TL and PC in 2M2B showed the highest synthesis rates and yields, but the yields were lower than those obtained with lactose. This difference may be due to the differing solubility of each sugar in the specific solvents as shown in FIG. 2. The rates obtained for all lipase/solvent combinations presented here are in the low mmol/h/g lipase range.

### Determining the Solubility of Lactose and Sucrose in Selected Solvents.

Referring now to FIG. 2, to determine the solubility of lactose and sucrose in various solvents, 0.05 g of each sugar was dissolved in 1.0 mL of water, MEK, acetone, acetonitrile, or 2M2B. These solutions were incubated at 55° C. for 3.5 h, and 900 μL of each were subsequently passed through a 0.2 micron filter. Aliquots, 600 μL, of each filtered sample were dried by a Savant SpeedVac system. The dry sugar in each tube was re-suspended in 600 μL de-ionized water. Aliquots, 20 μL, of each sample were analyzed by HPLC with water as the mobile phase and detected with an evaporative light scattering detector (ELSD) (Alltech ELSD 800) at 40° C. with a nitrogen gas pressure of 3.65 bar. The amount of sugar in each sample was determined by comparing peak areas to the lactose-in-water control.

Referring to FIG. 2 in more detail, there is shown the solubility of lactose and sucrose in MEK, acetone, acetonitrile and 2M2B. The solubility test was done with 5% sugar solutions to ensure complete solubility in water. Each sugar showed limited solubility in each solvent with the solubility in 2M2B being the highest at approximately 700-800 micromol/L solvent. The solubilities in the other solvents were much lower at 50-200 micromol/L solvent with sucrose about half as soluble as lactose. This difference in solubility may result in a generally higher LML synthesis than SML synthesis. The yield obtained for LML synthesis with PC in 2M2B is 56.6%; the amount of lactose solubilized over the synthesis time was greater than 50%, which is 100 times higher than the yield predicted based on the data in FIG. 2. Therefore, as the esters (LML or SML) are synthesized, the insoluble sugars solubilize to maintain equilibrium. The limiting factors in the synthesis and yield may be a combination of the sugar solubility and inactivation of the lipase.

Reactions with the highest rates of conversion allow more of the sugar to be solubilized and as a result the reactions have a higher yield. The higher solubility of lactose and sucrose in 2M2B resulted in the highest yields and synthesis rates, excluding LML synthesis by TL in acetone. If solubility were the only limiting factor, we would assume a higher or at least equal yield for SML in 2M2B with each lipase. But this was observed for only two of the four lipases (TL and CA). The sugar type and lipase specificity may also influence the rate of LML or SML synthesized.

### Nuclear Magnetic Resonance (NMR) and Mass Spectrometry Analysis

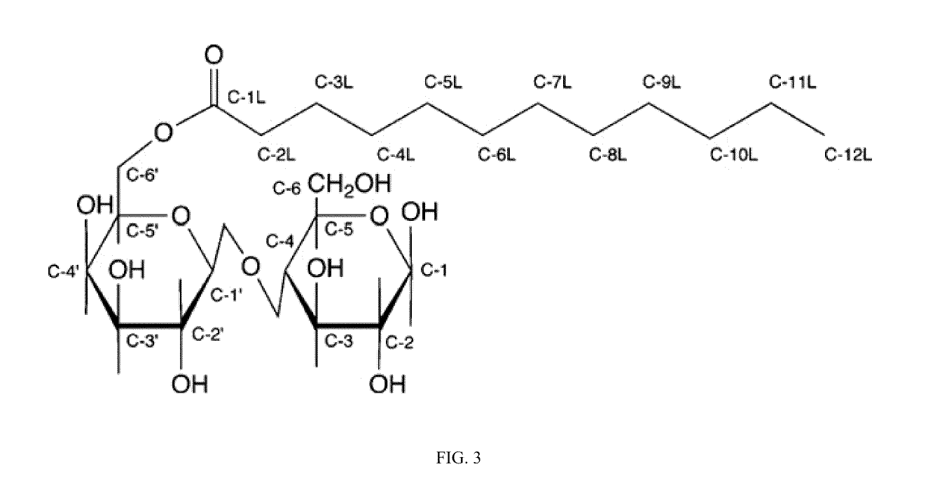

Referring now to FIG. 3, LML for NMR and mass spectrometry analyses was synthesized as described above and purified using C18 solid phase extraction columns (Alltech, Englewood, Colo., USA) for reactions catalyzed by TL in acetone and MM and PC in 2M2B. Columns were activated with 100% acetonitrile and rinsed with water. Reactions were added to the columns, the column washed with water and LML was eluted with 32% acetonitrile in water. Samples were analyzed by HPLC to confirm purity. 1H and ¹³C NMR spectra of LML dissolved in d6-DMSO were collected at 295K on a Bruker ARX-400 at 400 and 100 MHz, respectively. For comparison, the 1H and 13C NMR spectra of alpha-lactose, vinyl laurate, and lauric acid were collected under identical conditions. Chemical shifts (1) are referenced to the residual 1H (2.50 ppm) and 13C (39.50 ppm) resonances of d6-DMSO (99.9%). Mass spectrometry data was obtained at the Mass Spectrometry Facilities in the Departments of Chemistry at the University of California, Riverside, and the University of Utah. Samples were analyzed using either APCI or ESI ionization.

Again referring to FIG. 3, analysis of the 13C NMR features of the purified LML synthesized by TL, MM and PC revealed that the LML products were all esterified at the C6′ carbon with lactose primarily in the alpha configuration. The key indications of esterification at the C6′ position are: (1) the downfield shift of the C6′ 13C NMR resonance from 60.57 ppm in alpha-lactose to 63.30 ppm (Table 2) in LML, and (2) an upfield shift for the resonance of the adjacent C5′ carbon resonance in LML. The atom numbering scheme for LML is given in FIG. 3. The esterification of sucrose, a non reducing sugar, with lipids has been shown to occur most frequently in the C6 position but is dependent on the lipase type.

TABLE 2
¹³C {¹H} NMR resonances for α-lactose and α-C6′
lactose monolaurate ester.in d₆-DMSO at 295 K.

| Assignment^{a,b} | α-Lactose (ppm) | LML^{c} (ppm) |
|---|---|---|
| C-1′ | 103.86 | 103.58 |
| C-1 | 92.85 | 92.02 |
| C-4 | 81.34 | 81.21 |
| C-5′ | 75.48 | 72.77 |
| C-3′ | 73.22 | 72.38 |
| C-5 | 72.13 | 72.18 |
| C-3 | 71.36 | 71.24 |
| C-2′ | 70.60 | 70.27 |
| C-2 | 69.80 | 69.70 |
| C-4′ | 68.14 | 68.25 |
| C-6′ | 60.57 | 63.30 |
| C-6 | 60.38 | 60.43 |
| C-1L | N/A | 172.91 |
| C-2L | N/A | 33.28 |
| C-3L | N/A | 24.32 |
| C-4L to C-9L | N/A | 29.02^{d}, 28.91, 28.72^{d}, 28.49 |
| C-10L | N/A | 31.30 |
| C-11L | N/A | 22.11 |
| C-12L | N/A | 13.98 |

^{a}For atom numbering scheme see FIG. 3.
^{b}Assignment of sugar ring carbon resonances.
^{c}LML sample produced using the immobilized lipase from Thermomyces lanuginosus (TL). LML produced using the lipases from Psuedomonas cepacia (PC) and Mucor miehei (MM) produced similar ¹³C NMR features. In selected samples an α/β mix of sugars was present.
^{d}Signal intensity indicates the overlap of two resonances.
Mass spectrometry analysis of the LML produced using lipases from TL, PC, and MM gave a molecular ion peak at m/z 547, which is consistent with the formulation [NaLML] + and the monoesterification of lactose.

### Reaction Rates and Yields

Still referring to FIG. 3, LML fractions were collected from the HPLC runs using a fraction collector and were dried with a Speed-Vac and the mass measured. This dry mass was resuspended in 40:60 acetonitrile:water and serial dilutions were analyzed via HPLC to form a standard curve (mg/peak area). The standard curve was used to calculate the mg/mL of LML produced each day by each reaction and was plotted against days. A line of best fit was plotted until the maximum day of monoester production and the slope of this graph gave the rate of the reaction reported as mmol/h/g lipase. Each reaction vial contained 42 mM (or 0.13 mmol in 3 mL) of either lactose or sucrose which acted as the limiting substrate. The molecular weight of LML product was determined to be 547 g/mol, which gives a maximum theoretical yield of 22 mg/mL. Measured monoester amounts were compared to this number to give actual yield.

### Enzymatic Reactions and High-Performance Liquid Chromatography (HPLC)

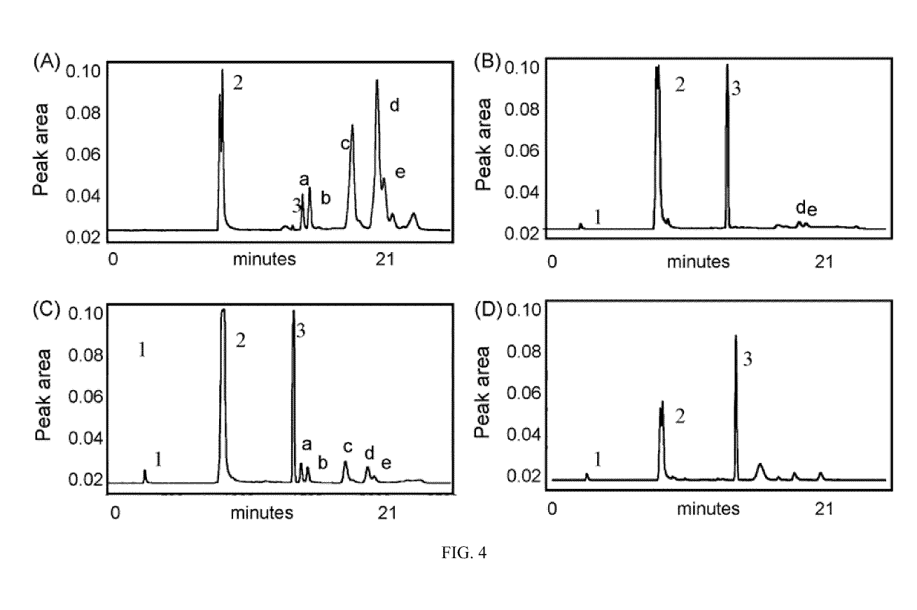

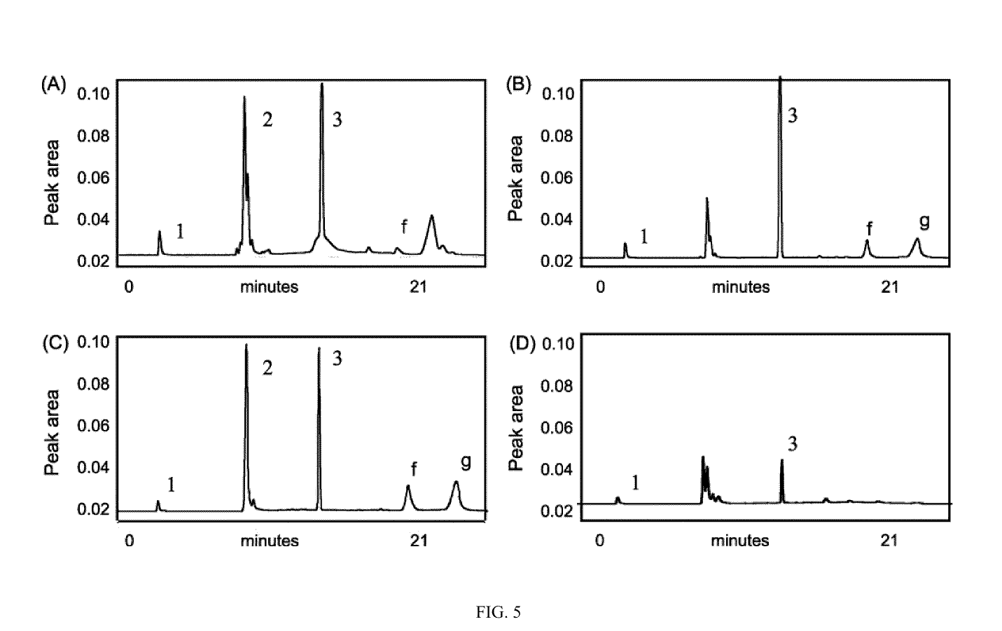

Referring now to FIGS. 4 and 5, prior to assembling reactions, solvents (acetone, MEK, 2M2B) were dried overnight in a room temperature shaker with molecular sieves (0.1 g/mL). Reactions were assembled in 4 mL glass vials with Teflon caps. Solvent (3 mL) was added to sugar (44.16 mg or 42 mM), immobilized lipase (0.068 g) and molecular sieves (10%). Vials were inverted several times, and vinyl laurate (0.128 mg or 0.13M) was added which resulted in a 1:3 molar ratio of sugar:vinyl laurate. Vials were placed at 55° C. in an orbital shaker. Aliquots were removed from each vial daily for HPLC analysis.

Again referring to FIG. 4, analysis of the reactions was performed at room temperature by HPLC (Beckman System Gold 125 Solvent Module) equipped with a Luna 5 micron C18 (2) 100 Å column (250 mm×4.6 mm, Phenomenex, Torrance, Calif., USA). The mobile phase consisted of a gradient from 10% acetonitrile:water (40:60) to 100% acetonitrile:water (95:5), with a flow rate of 1.0 mL/min over 24 minutes. Products and standards were detected with an ELSD at 60° C. with a nitrogen gas pressure of 3.65 bar. Standards consisted of lactose, sucrose, lauric acid and vinyl laurate.

FIGS. 4 (lactose reactions) and 5 (sucrose reactions) show HPLC chromatograms of the products synthesized for representative reactions. In both figures, peaks that have been identified include lactose or sucrose, SML or LML and lauric acid. In each chromatogram in which the solvent was 2M2B (all except FIG. 4A), there is a sugar peak present, which supports the lactose solubility data in FIG. 2. Depending on the lipase used, there are multiple products present that have greater hydrophobicity (e.g. retention times) than lauric acid. We assume these are sugar esters with multiple lauric acids esterified. The greatest number of these products is present in reactions with lactose as the substrate with TL, followed by reactions with either lactose or sucrose with PC, MM and CA. Peaks with the same letter among the chromatograms have the same retention times and may be similar esters with multiple lauric acids esterified. Doublet LML peaks were observed for most lipase/solvent reactions except with the lipase from PC. The doublet peaks are presumably from the lactose in the alpha and beta configurations while the doublet peaks for the SML are presumably from the presence of both the C6 and C6′ products. The data in FIG. 1A shows that some of the lipase/substrate combinations exhibit a decrease in yield over time. Specifically, reactions involving MM in 2M2B and acetone, TL in acetone, and PS in acetone showed a decrease in yield. It is possible that the monoester LML is being converted to di- or multi-ester sugar products for reactions that are synthesized by TL and PC since the chromatographs for these lipases show multiple hydrophobic products. This is probably not the case for reactions with MM since the chromatograms show limited multi-ester peaks. It is why the yield decreases over time with this lipase. There was no obvious decrease in any of the yields in the sucrose reactions in FIG. 1B.

### Response Surface Analysis

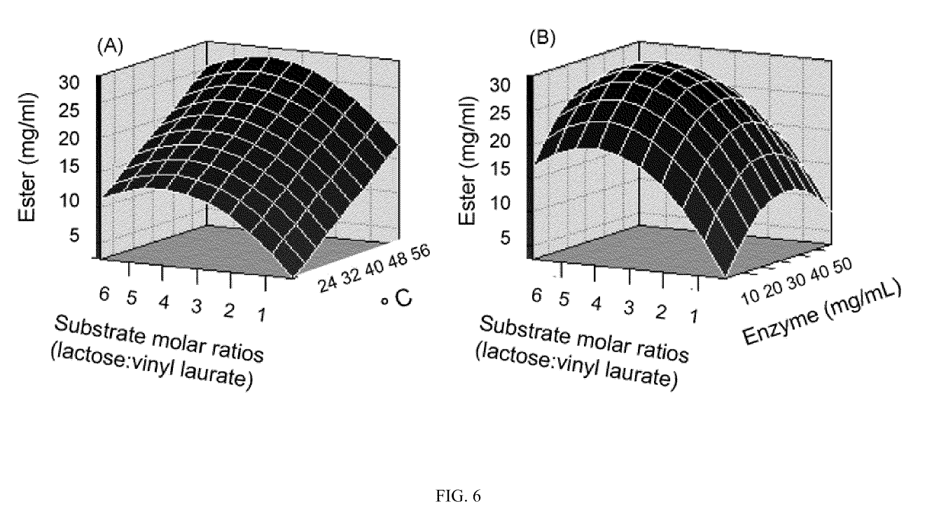

Referring now to FIG. 6, a response surface design (Roquemore R311A hybrid, Statistical Analysis System) with three factors (temperature, lipase concentration, and lactose:vinyl laurate ratio) was conducted with MM in 2M2B to determine the optimal conditions for LML synthesis. The factor levels were 25-55° C. for temperature, 10-50 mg/mL lipase, and 1-5 for the ratio of lactose to vinyl laurate. This resulted in 11 design points, including one center point. The average LML yield for each design point in duplicate was analyzed by regression to fit a second-order polynomial equation. The ridge max option was used to compute the estimated ridge of maximum response for increasing radii from the center of the original design. This resulted in the optimal synthesis conditions.

Still referring to FIG. 6, the RSM analysis was conducted for the synthesis of LML using MM in 2M2B because this combination resulted in a high yield, faster rate, and the lipase is more economical than the others. The experimental design and concentration of LML synthesized at each design point are given in Table 3.

TABLE 3
Response surface design and experimental results.

| | | Lipase (MM) | | |
|---|---|---|---|---|
| | Temperature | amount | Substrate molar ratios | LML yield |
| Run | (° C.) | (mg/mL) | (lactose:vinyl laurate) | (mg/mL) |
| 1 | 40 | 30 | 1:5.83 | 11.00 |
| 2 | 40 | 30 | 1:0.17 | 0.43 |
| 3 | 25 | 10 | 1:0.17 | 2.13 |
| 4 | 55 | 10 | 1:4.41 | 21.43 |
| 5 | 25 | 50 | 1:4.41 | 4.22 |
| 6 | 55 | 50 | 1:4.41 | 21.33 |
| 7 | 61 | 30 | 1:1.59 | 22.00 |
| 8 | 18 | 30 | 1:1.59 | 1.70 |
| 9 | 40 | 58.28 | 1:1.59 | 4.02 |
| 10 | 40 | 1.72 | 1:1.59 | 0.81 |
| 11 | 40 | 30 | 1:3 | 13.95 |

Among the various treatments, the highest yields were obtained with runs 4, 6 and 7, while runs 2 and 10 showed the lowest yields. ANOVA results revealed that all three variables and the interactions of temperature×temperature and ratio×ratio exhibited statistically significant effects (p <0.05) on the yield of LML. The estimate response model equation, without the insignificant variables, was used to estimate the enzymatic synthesis of LML with MM and is as follows: Y=−353.78+5.81 X₁+6.9 X₂+101.13 X₃−0.11 X₂X₂−13.50 X₃X₃ (1) where Y is the response factor in peak area and X₁, X₂, and X₃ are the independent factors of temperature, lipase concentration (mg/mL) and ratio of lactose to vinyl laurate. The coefficient of determination (R2) was 0.95 indicating that the model was suitable to represent the factors. Canonical analysis of the three variables determined that the most critical factor was temperature, with the concentration of lipase being the second most influential factor on the yield. FIG. 6 shows the effect of ratio, temperature and lipase concentration on the amount of LML synthesized. The stationary point for maximum yield was determined to be a saddle point; therefore there was no unique optimum. This can be seen in FIG. 6 where there is a narrow range of ratios (3.7-3.8) at 61° C. that gives maximum LML yield. FIG. 6 also shows the influence of temperature on yield is linear, with increasing yields with an increase in temperature while the influences of substrate ratio and lipase concentration have narrow optimum values. Ridge maximum analysis was conducted, which determines the optimal reaction conditions with the maximum, predicted yield. The conditions of 61° C., 32 mg/mL of lipase and a lactose:vinyl laurate ratio of 1:3.8 was predicted to yield 28 mg/mL LML. Our experimental results were in agreement with a concentration of 27.8 mg/mL obtained with conditions listed above. Therefore RSM was successful in determining the optimal conditions for LML synthesis in 2M2B with MM.

### Microbial Inhibition

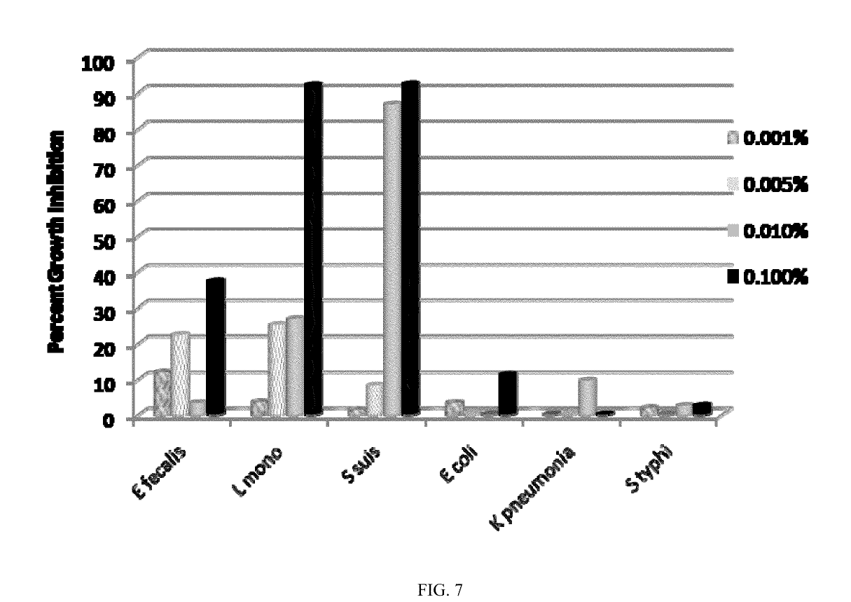

Referring now to FIG. 7 and Table 4, the microbial inhibitory characteristics of LML synthesized and purified as described above, were investigated against Enterococcus faecalis (ATCC 700802), Listeria monocytogenes, Staphyloccus suis, Escherichia coli H7P:0157H7 (ATCC 35150), Salmonella typhimurium (ATCC 700720) and Klebsiella pneumoniae (ATCC 700721). Cultures were grown in appropriate microbial media with antibiotics and diluted to 10⁵ colony forming units (CFU) per mL. Cultures (0.5 mL or 10^{2.5} CFU total) were added to microtiter wells and an initial optical density (OD) at 600 nm was recorded. For treatments, LML in concentrations ranging from 0.001 to 0.1% was added to individual microtiter wells and the OD was again measured after 48 hours. Controls were treated similarly, but without the addition of LML. Percent growth inhibition was determined by comparing the OD reading for the controls and treatments.

FIG. 7 shows that the gram positive bacteria (E. feacalis, L. monocytogenes, and Staphyloccus suis) were inhibited by LML at concentrations of 0.1% (1 mg/mL) with limited inhibition at LML concentrations of 0.005% and less. The gram negative bacteria exhibited minimal susceptibility to inhibition by LML.

TABLE 4
Microbes used to test inhibitory characteristics of LML

| | | | | | Growth | |
|---|---|---|---|---|---|---|
| | | | Gram | | Condition | Antibiotic |
| Microorganism | Designation | ATCC # | Reaction | Media | (rpm/° C.) | Resistance |
| Enterococcus | V583 | 700802 | Positive | BHI | 220/37 | Rifampicin |
| faecalis | | | | | | |
| Leisteria | EGDe* | N/A | Positive | BHI | 220/37 | Penicillin G |
| monocytogenes | | | | | | |
| Streptococcus | 89/1597* | N/A | Positive | BHI | 220/37 | Penicillin G |
| suis | | | | | | |
| Escherichia coli | EDL 931 | 35150 | Negative | LB | 220/37 | Polymyxin B |
| H7P:0157 | | | | | | |
| Salmonella | N/A | 700720 | Negative | LB | 220/37 | Polymyxin B |
| typhimurium | | | | | | |
| Klebsiella | N/A | 700721 | Negative | LB | 220/37 | Polymyxin B |
| pneumoniae | | | | | | |

*denotes that these are not ATCC (provided by the lab of Dr. Bart Weimer, U. C. Davis)

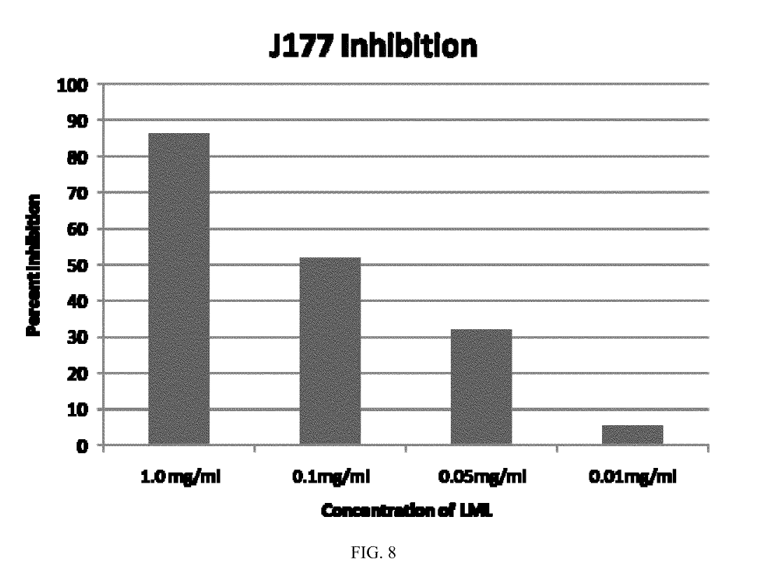

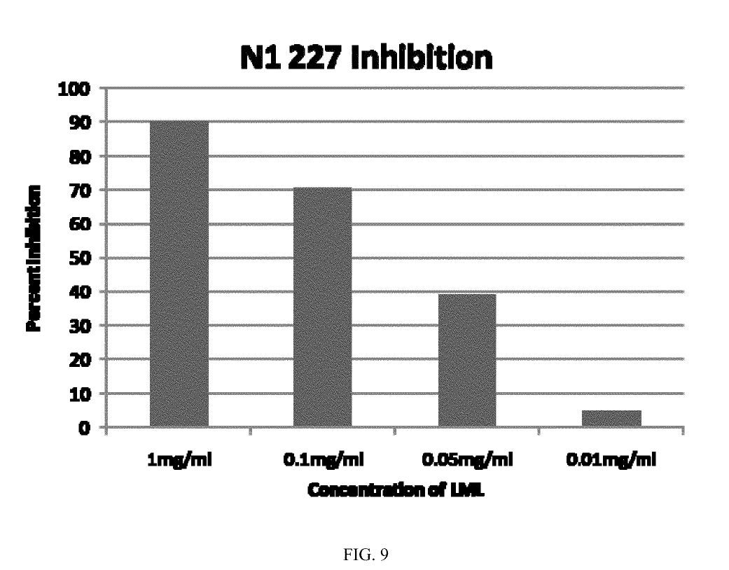

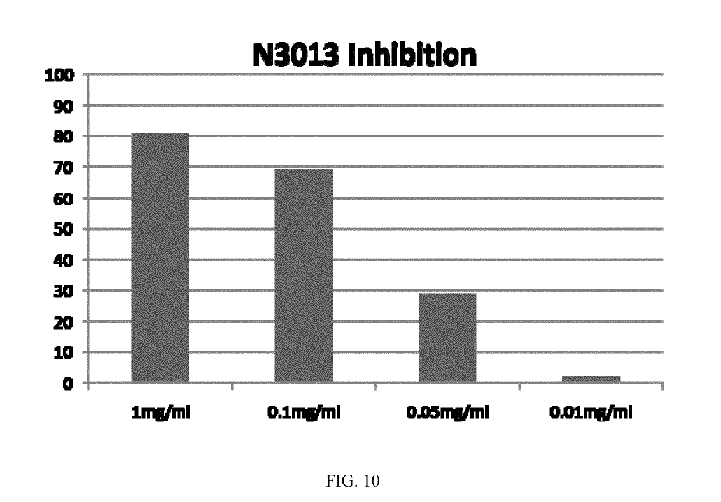

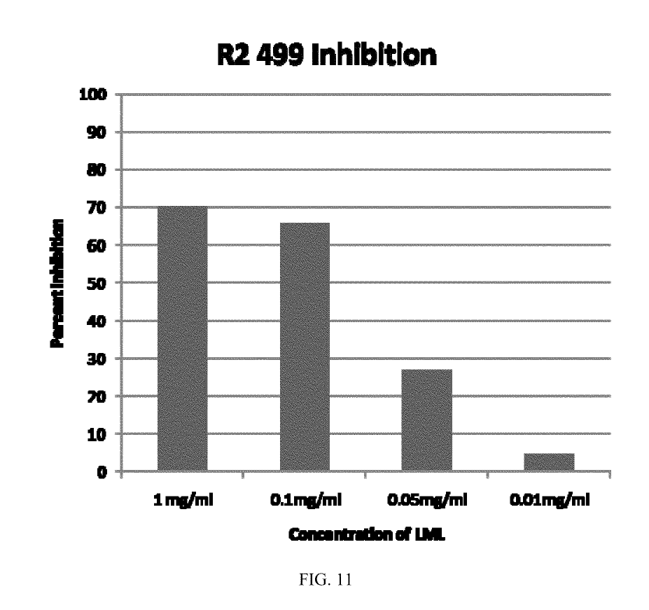

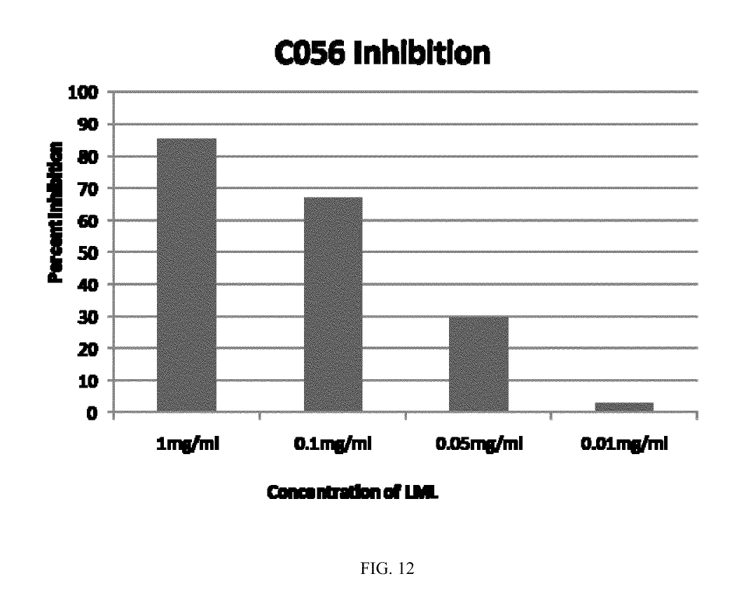

Referring now to FIGS. 8 through 12, in light of the microbial inhibitory effect shown with Listeria monocytogenese, we obtained clinical isolates of Listeria monocytogenes from the International Life Science Institute Database, Cornell University and tested LM at the same concentrations as listed above with the clinical isolate. Inhibitory effects against clinical isolates of Listeria monocytogenese are shown in FIGS. 8, 9, 10, 11 and 12 (inhibition shown is percent growth inhibition). The clinical isolates are described in Table 5.

TABLE 5
Description of Clinical Isolates

| | Where isolated from (and time of |
|---|---|
| Clinical Isolate | isolation, if known) |
| FSL Jl-177; ribotype DUP-1051D; | Isolated from human sporadic case |
| lineage I; serotype ½b | |
| FSL C1-056; ribotype DUP-1030A; | Isolated from human sporadic case |
| lineage II; serotype ½a | |
| FSL N3-013; ribotype DUP-1042B; | Food isolate associated with human |
| lineage I; serotype 4b | listeriosis epidemic in the UK |
| | (1988-1990) |
| FSL R2-499; ribotype DUP-1053A; | Human isolate associated with US |
| lineage II; serotype ½a | outbreak linked to sliced turkey |
| | (2000) |
| FLS N1-227; ribotype DUP-1044A; | Food isolate associated with US |
| lineage I; serotype 4b | outbreak (1998-1999) |

Still referring to FIGS. 8 through 12, microbial inhibitory studies were carried out in microtitre well plate method. Cultures were grown in appropriate microbial media with antibiotics and diluted to 10⁵ colony forming units (CFU) per mL. Cultures (0.5 mL or 10^{2.5} CFU total) were added to microtiter wells and an initial optical density (OD) at 600 nm was recorded. The studies were conducted by adding different concentrations of LML as described above with appropriate controls (media only, media plus cells, media plus cells and 0.1% Polysorbate 80, same amount of ethanol as in the vol of LML plus 0.1% polysorbate 80 with cells). Plate counts of all controls and treatments were done. For each strain and treatment, the experiments were done 6 times and replicated at least once. Data shown has a coefficient of variation of less than 10%. The growth in the control (with ethanol and tween) was compared to the treatments to give the percent inhibition shown in the graphs. Plate counts done with the treatments showed that the type of inhibition was bacterial static, limited growth occurred with treatments compared to the controls. Each strain of Listeria monocytogenese used was 70-90% inhibited by LML at concentrations of 1.0 mg/mL.

Polysorbate 80, which is a non-ionic emulsifier (commercially known as tween 80) was used to ensure that the LML remained in solution during the microbial inhibitory studies. Polysorbate 80 in the concentration of 0.1% is food grade.

### EXAMPLES

The above disclosure provides for multiple examples and embodiments for the present invention.

In one such example there is provided a LML compound with the structure shown in FIG. 3. LML has disclosed utility as an antimicrobial agent, and will likely also possess utilities commonly associated with sugar esters. Thus, examples related to the LML compound of FIG. 3 would include antimicrobial compositions. In one such example, LML may be provided in the form of a surface decontaminant, in a composition comprising LML, a diluent, and other minor components. The relative proportions of LML and diluents may be adjusted such that the concentration of LML is substantially the same as the concentrations shown in this application to inhibit or prevent microbial growth. The surface decontaminant may provide a sanitizing effect. Minor components of the surface decontaminant may include stabilizing agents and other antimicrobial agents. Minor components may also include dyes or pigments, skin conditioners, emulsifiers, and wetting agents. The surface decontaminant would be useful in decontaminating many surface types, including, but not limited to, household kitchen surfaces and other food preparation surfaces. LML can be synthesized using food grade reactants and thus may be useful is decontaminating the surface of food products. The antimicrobial activity of LML against Listeria monocytogenes suggests the surface of meat products would be a particularly favorable use of LML as an antimicrobial decontaminating agent. Related examples may include methods of inhibiting, preventing, reducing or eliminating the presence or growth of a microorganism on a surface. Such methods would involve contacting the surface with an antimicrobial composition containing a sufficient amount of LML at a sufficient concentration and for a sufficient period of time to inhibit, prevent, reduce or eliminate the presence or growth of a microorganism susceptible to the antimicrobial activity of LML.

In another example, LML may be provided in a composition useful in emulsification of personal care products for the cosmetic industries. LML may find uses as an emulsifier, surfactant or lipid phase modifier, especially as an alternative to sucrose esters or other sugar esters.

In another example, LML is synthesized by (i) providing a first substrate, a second substrate, a solvent and an immobilized lipase, wherein the first substrate is lactose, and the second substrate is lauric acid, vinyl laurate, or a combination of lauric acid and vinyl laurate, (ii) contacting the first substrate and the second substrate to the immobilized lipase in the presence of the solvent, wherein the contacting occurs in a nonaqueous mixture, and wherein the contacting may optionally occur in the presence of molecular sieves, (iii) allowing the mixture to undergo biochemical reaction and form a reacted mixture at a temperature below the evaporation point of the solvent, wherein one product of the reaction is LML, (iv) filtering the reacted mixture with a filter capable of removing a substantial amount of the immobilized lipase, unreacted first substrate, unreacted second substrate, and any optionally included molecular sieves, (iv) drying the reacted mixture, (v) resuspending the reacted mixture in a solution comprising ethanol (other alcohols might also be used), wherein the resuspending may result in the formation of a solution phase and a lipid phase, and wherein the resuspending may result in some precipitation of unreacted first substrate, and may also result in unreacted second substrate in the lipid phase, and wherein most of the LML product is within the solution phase, (vi) substantially separating the solution phase from the precipitated first substrate and second substrate, and also separating the solution phase from the lipid phase, such that a solution phase comprising LML is substantially isolated,

(vii) optionally confirming the purity of the LML in the solution phase by HPLC. In related examples specific immobilized lipases, solvents, and reaction conditions can be combined and used to produce LML, and RSM may be used to optimize the production of LML.

I claim:

1. A chemical compound, comprising, the following compound:

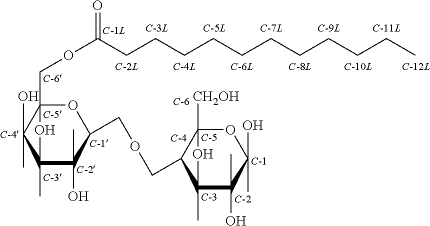

2. A method of synthesizing the compound of claim 1, comprising:
(i) providing a first substrate, a second substrate, a solvent and an immobilized lipase, wherein the first substrate is lactose, and the second substrate is lauric acid, vinyl laurate, or a combination of lauric acid and vinyl laurate,
(ii) contacting the first substrate and the second substrate to the immobilized lipase in the presence of the solvent, wherein the contacting occurs in a nonaqueous mixture, and wherein the contacting may optionally occur in the presence of molecular sieves,
(iii) allowing the mixture to undergo biochemical reaction and form a reacted mixture at a temperature below the evaporation point of the solvent, wherein one product of the reaction is LML,
(iv) filtering the reacted mixture with a filter capable of removing a substantial amount of the immobilized lipase, unreacted first substrate, unreacted second substrate, and any optionally included molecular sieves,
(iv) drying the reacted mixture,
(v) resuspending the reacted mixture in a solution comprising an alcohol, wherein the resuspending may result in the formation of a solution phase and a lipid phase, and wherein the resuspending may result in some precipitation of unreacted first substrate, and may also result in unreacted second substrate in the lipid phase, and wherein most of the LML product is within the solution phase,
(vi) substantially separating the solution phase from the precipitated first substrate and second substrate, and also separating the solution phase from the lipid phase, such that a solution phase comprising LML is substantially isolated,
(vii) optionally confirming the purity of the LML in the solution phase by HPLC.

3. The method of claim 2, wherein said temperature is kept between 50° C. and 70° C.

4. The method of claim 2, wherein said immobilized lipase is one or more lipase or lipases selected from a group comprising TL, MM, PC, or CA.

5. The method of claim 2, wherein said solvent is one or more solvents selected from a group comprising 2M2B, acetone, or MEK.

6. The method of claim 2, wherein an initial concentration of lactose is sufficiently high such that at least a substantial amount of the lactose is an insolubilized lactose, and wherein the synthesis of LML results in solubilization of the insolubilized lactose, wherein the solubilization of the insolubilized lactose contributes to the overall yield of LML.

7. The method of claim 2, wherein said immobilized lipase is MM and wherein the concentration of the MM is between 1.72 mg/ml and 50 mg/ml, and wherein said solvent is 2M2B, and wherein said temperature is kept between 18° C. and 61° C., and wherein the ratio of lactose to vinyl luarate is between 1:0.17 and 1:5.83.

8. The method of claim 2, wherein the limiting reactant is lactose.

9. The method of claim 2, wherein said immobilized lipase is TL and said solvent is acetone.

10. The method of claim 2, wherein said immobilized lipase is one or more lipase or lipases selected from a group comprising TL, MM, PC, or CA, and wherein said solvent is one or more solvents selected from a group comprising 2M2B, acetone, of MEK.

11. The method of claim 10, wherein said temperature is kept between 50° C. and 70° C.

12. The method of claim 10, wherein said immobilized lipase is one or more lipase or lipases selected from a group comprising TL, MM, PC, or CA.

13. The method of claim 10, wherein said solvent is one or more solvents selected from a group comprising 2M2B, acetone, of MEK.

14. The method of claim 10, wherein an initial concentration of lactose is sufficiently high such that at least a substantial amount of the lactose is an insolubilized lactose, and wherein the synthesis of LML results in solubilization of the insolubilized lactose, wherein the solubilization of the insolubilized lactose contributes to the overall yield of LML.

15. The method of claim 10, wherein said immobilized lipase is MM and wherein the concentration of the MM is between 1.72 mg/ml and 50 mg/ml, and wherein said solvent is 2M2B, and wherein said temperature is kept between 18° C. and 61° C., and wherein the ratio of lactose to vinyl luarate is between 1:0.17 and 1:5.83.

16. The method of claim 10, wherein the limiting reactant is lactose.

17. The method of claim 10, wherein said immobilized lipase is TL and said solvent is acetone.

18. A method of inhibiting, preventing, reducing or eliminating the presence or growth of a microorganism on a surface, comprising: contacting the surface with an antimicrobial composition comprising, a sufficient amount of LML at a sufficient concentration and for a sufficient period of time to inhibit, prevent, reduce or eliminate the presence or growth of a microorganism susceptible to the antimicrobial activity of LML.

19. The method of claim 20, wherein said gram positive bacteria is selected from a group comprising: Enterococcus faecalis, Listeria monocytogenes and Streptococcus suis.

20. The method of claim 20, wherein the surface further comprising the surface of a food product.
